(19) United States
(12) Patent Application Publication
(10) Pub. No.: US 2009/0305952 A1
(43) Pub. Date: Dec. 10, 2009

(54) Anionic polyamide functionalized with a tryptophan unit

(75) Inventor: Remi Soula, Lyon (FR)

Correspondence Address: OLIFF & BERRIDGE, PLC, P.O. BOX 320850, ALEXANDRIA, VA, 22320-4850, US

(73) Assignee: ADOCIA, Lyon (FR)

(21) Appl. No.: 12/457,096

(22) Filed: Jun. 1, 2009

Related U.S. Application Data
(60) Provisional application No. 61/129,014, filed on May 30, 2008.
(60) Provisional application No. 61/129,619, filed on Jul. 8, 2008.

(30) Foreign Application Priority Data
Jul. 7, 2008 (FR) 08 54620

(51) Int. Cl. A61K 38/14; C07K 14/00; A61K 47/42; A61K 38/16; A61K 38/02; A61P 43/00

(52) U.S. Cl. 514/8; 530/402; 514/773; 514/12; 514/2

(57) ABSTRACT

The invention relates to an anionic polyamide functionalized with at least one tryptophan unit, -Trp, said tryptophan unit being linked to the polyamide by an amide function separated from the backbone by a linker arm, the polyamide being a chain selected from the group constituted of the following polymers:

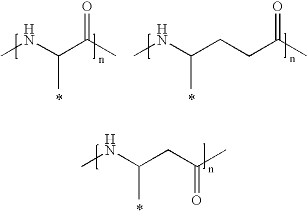

L being selected from the group constituted of a single bond, a —CH₂— group or a —CH₂CH₂— group, -Trp being an L and/or D tryptophan residue produced from the coupling between the amine of the tryptophan or of a tryptophan derivative, selected from the group constituted of tryptophanol, tryptophanamide and alkali-metal cation salts thereof, and an acid borne by the polyamide. The present invention also relates to a pharmaceutical composition containing one of the polyamides according to the invention.

The present invention relates to novel biocompatible polymers based on anionic polyamino acids or anionic polyamides.

These polymers provide an advantage, in particular, for the administration of active ingredient(s) (Al(s)) to humans or to animals for therapeutic and/or prophylactic purposes. In this use, the polymers used should have good biocompatibility.

The amphiphilic nature of anionic polyamides functionalized with hydrophobic groups has been described, as has the use thereof for the formulation of active ingredients and of vaccines.

Application US20070178126 describes poly(alpha-glutamate)s bearing mono-, di-, tri- or tetraamino acids selected from alanine, valine, leucine, isoleucine and phenylalanine.

Patent application US20070160568 describes polyglutamates and polyaspartates bearing lysine and ornithine derivatives which have been hydrophobized.

Patent application FR2840614 describes polyglutamates and polyaspartates bearing an alpha-tocopherol unit.

In the article Biomaterials 28 (2007) 3427-3436, Akashi et al. describe poly(gamma-glutamate)s functionalized with amino acid esters. Phenylalanine ethyl ester, tryptophan methyl ester and tyrosine ethyl ester are grafted onto pendent acid functions.

EP0685504 describes polyaspartic acid derivatives and the method for preparing same, said derivatives comprising at least one monomer comprising an amino acid residue, it being possible for said amino acid to be tryptophan or a tryptophan ester.

These monomers are obtained by opening of the rings of a polysuccinimide through the action of an amino acid ester; these polymers cannot therefore comprise polyamide monomers that are salified and not substituted with an amino acid residue; in fact, when the ring is not open, the polymer comprises a cyclic monomer.

Surprisingly, novel anionic polyamino acid derivatives functionalized with at least one indole unit of natural origin are easy to synthesize by a method which does not use carbodiimides and which has the same advantages in terms of formulation.

The present invention relates to novel anionic polyamino acid derivatives functionalized with at least one indole unit of natural origin which make it possible to meet the criteria of amphiphilicity and biocompatibility.

These novel polyamino acids comprising an indole unit are selected from biocompatible polyamides functionalized with tryptophan or a natural derivative of tryptophan.

The invention therefore relates to an anionic polyamide functionalized with at least one tryptophan unit, denoted -Trp, said tryptophan unit being linked to the polyamide by an amide function separated from the chain by a linker arm L,
the polyamide being a chain selected from the group constituted of the following polymers,

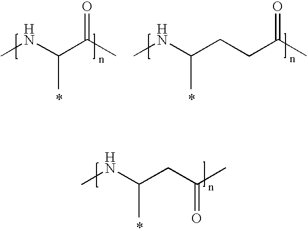

L being selected from the group constituted of a single bond, a —CH₂— group or a —CH₂CH₂— group,
-Trp being an L and/or D tryptophan residue produced from the coupling between the amine of the tryptophan or of a tryptophan derivative, selected from the group constituted of tryptophanol, tryptophanamide and alkali-metal cation salts thereof, and an acid borne by the polyamide.

The expression “linked to the polyamide by an amide function separated from the chain by a linker arm L” is intended to mean a bond forming an amide function between the amine function of the tryptophan or of the tryptophan derivative and the carboxyl function borne by the polyamide before the grafting reaction.

According to the invention, the tryptophan residue -Trp corresponds to the following formulae:

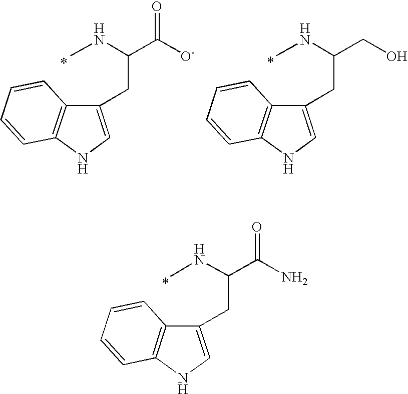

According to the invention, the functionalized anionic polyamide can correspond to general formula I below:

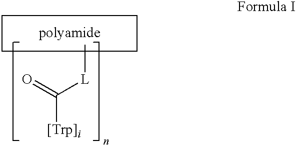

the polyamide, L and -Trp having the meanings given in the definitions of the invention above, and
n is the number of acid functions borne by the polyamide and is between 10 and 10000,
i is the molar fraction of acids substituted with -Trp and is between 0.05 and 0.6.

When the acid is not substituted with -Trp, then the acid is a carboxylate of a cation, preferably a cation of an alkali metal such as Na or K.

It corresponds more particularly to general formula I′ below:

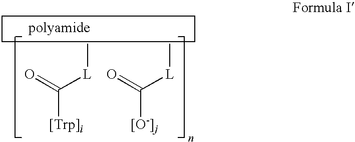

the polyamide, L and -Trp having the meanings given in the definitions of the invention above, and
n is the number of acid functions borne by the polyamide and is between 10 and 10000,
i is the molar fraction of acids substituted with -Trp and is between 0.05 and 0.6, and
j is the molar fraction of acids not substituted with -Trp and j is equal to n-i,

when the acid is not substituted with -Trp, then the acid is a carboxylate of a cation, preferably a cation of an alkali metal such as Na or K.

In one embodiment, the invention relates to an anionic polyamide functionalized with at least one tryptophan unit, -Trp, said tryptophan unit being linked to the polyamide by an amide function separated from the backbone by a linker arm L, characterized in that it corresponds to formula I′ below:

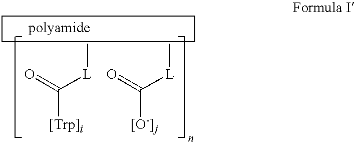

the polyamide being a chain selected from the group constituted of the following polymers

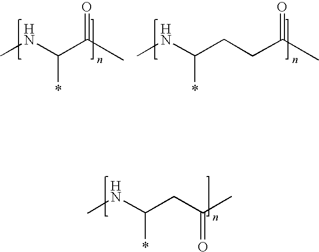

L being selected from the group constituted of a single bond, a —CH₂— group or a —CH₂CH₂— group,
-Trp being an L and/or D tryptophan residue produced from the coupling between the amine of the tryptophan or of a tryptophan derivative, chosen from the group constituted of tryptophanol, tryptophanamide and alkali-metal cation salts thereof, and an acid borne by the polyamide,
n is the number of acid functions borne by the polyamide and is between 10 and 10000,
i is the molar fraction of acids substituted with -Trp and is between 0.05 and 0.6, and
j is the molar fraction of acids not substituted with -Trp and j is equal to n-i,

when the acid is not substituted with -Trp, then the acid is a carboxylate of a cation, preferably a cation of an alkali metal such as Na or K.

In one embodiment, the number of acid functions n is between 10 and 1000.

In another embodiment, the number of acid functions n is between 10 and 500.

In one embodiment, the polyamide may be selected from the poly(alpha-glutamate)s having the formula below:

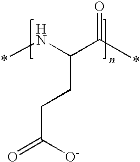

In one embodiment, the polyamide may be selected from the poly(gamma-glutamate)s having the formula below:

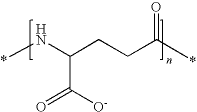

In one embodiment, the polyamide may be selected from the poly(alpha-aspartate)s having the formula below:

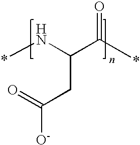

In one embodiment, the polyamide may be selected from the poly(beta-aspartate)s having the formula below:

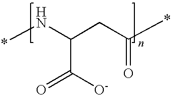

In one embodiment, the polyamide may be a poly(alpha,beta-aspartate).

The products according to the invention are obtained by means of a coupling method that is conventional in the field of peptide synthesis using chloroformates. This synthesis avoids the use of carbodiimides, which are toxic and expensive compounds.

The invention also relates to a pharmaceutical composition comprising one of the polyamino acids according to the invention as described above, and at least one active ingredient.

The term “active ingredient” is intended to mean a product in the form of a single chemical entity or in the form of a combination having physiological activity. Said active ingredient may be exogenous, i.e. it is provided by the composition according to the invention. It may also be endogenous, for example the growth factors that will be secreted in a wound during the first phase of healing and that may be retained on said wound by the composition according to the invention.

The invention also relates to a pharmaceutical composition according to the invention as described above, characterized in that the active ingredient is selected from the group constituted of proteins, glycoproteins and peptides.

According to the invention, the proteins or glycoproteins are selected from hormones such as insulin, hGH, from growth factors such as members of the transforming growth factor-β (TGF-β) superfamily, for instance bone morphogenic proteins (BMPs), platelet derived growth factors (PDGFs), insulin growth factors (IGFs), nerve growth factors (NGFs), vascular endothelial growth factors (VEGFs), or fibroblast growth factors (FGFs), and cytokines of the interleukin (IL) or interferon (IFN) type.

According to the invention, the non-peptide therapeutic molecules are selected from the group constituted of anticancer agents such as taxol or cis-platin.

According to the invention, the active ingredient is selected from the group of peptides selected from leuprolide or short sequences of parathyroid hormone (PTH).

The invention also relates to a pharmaceutical composition according to the invention as described above, characterized in that it can be administered orally, nasally, vaginally or buccally.

The invention also relates to a pharmaceutical composition according to the invention as described above, characterized in that it is obtained by drying and/or lyophilization.

The invention also relates to a pharmaceutical composition according to the invention as described above, characterized in that it can be administered in the form of a stent, of a film or coating of implantable biomaterials, or of an implant.

The pharmaceutical compositions are either in liquid form, or in the form of a powder, an implant or a film.

In the case of topical and systemic releases, the methods of administration envisaged are intravenous, subcutaneous, intradermal, intramuscular, oral, nasal, vaginal, ocular, buccal, etc., administration.

The invention also relates to the use of the functionalized polyamino acids according to the invention, for the preparation of pharmaceutical compositions as described above.

### EXAMPLE 1

### Synthesis of a Poly(Sodium Alpha-Glutamate) Modified with the Sodium Salt of Tryptophan

2 g of poly(sodium alpha-glutamate) having a weight-average molar mass of between 15 and 50 kg/mol (Fluka) and an average degree of polymerization n of approximately 200 is solubilized in water at 10 mg/g. The solution is passed through a Purolite resin so as to obtain poly(alpha-glutamic acid), which is subsequently lyophilized for 12 h. 1 g of poly(alpha-glutamic acid) is solubilized in 29 ml of DMF at 60° C. The solution is subsequently cooled to 0° C. prior to the addition of 0.85 mg of NMM in solution in 1.6 ml of DMF and of 0.91 g of ethyl chloroformate. After reaction for 10 minutes, 0.77 g of tryptophan is added to the opalescent solution. The suspension obtained is subsequently stirred for 60 minutes at 10° C.

An aqueous solution of imidazole (0.5 g in 2 ml of water) is then added, and the temperature is fixed at 30° C. The reaction medium is subsequently diluted in 40 ml of water. The final solution is placed in dialysis tubes (cutoff threshold of 8 kD) and is rinsed three times against 5 l of 0.9% NaCl and twice against 5 l of distilled water.

The polymer obtained has a random distribution of the tryptophan groups on the pendent acid functions. The structure can be represented diagrammatically in the following way

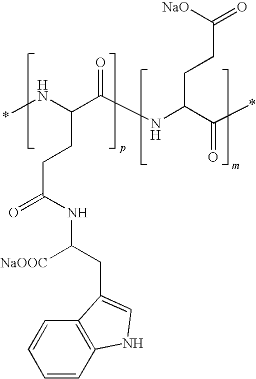

The molar fraction of tryptophan-modified acids is 0.4 according to the ¹H NMR D₂O (i=0.4). The number p of acid functions bearing a tryptophan is approximately 80. The number m of sodium carboxylate functions is approximately 120. The degree of polymerization n is the sum of m and p.

### EXAMPLE 2

### Synthesis of a Poly(Sodium Aspartate) Modified with the Sodium Salt of Tryptophan

2 g of poly(sodium aspartate) having a weight-average molar mass of between 15 and 50 kg/mol (Sigma) and an average degree of polymerization n of approximately 220 is solubilized in water at 10 mg/g. The solution is passed through a Purolite resin so as to obtain poly(aspartic acid), which is subsequently lyophilized for 12 h. 1 g of poly(aspartic acid) is solubilized in 29 ml of DMF at 60° C. The solution is subsequently cooled to 0° C. prior to the addition of 0.95 mg of NMM in solution in 2 ml of DMF and of 1.02 g of ethyl chloroformate. After reaction for 10 minutes, 0.86 g of tryptophan is added to the opalescent solution. The suspension obtained is subsequently stirred for 60 minutes at 10° C.

An aqueous solution of imidazole (0.58 g in 2 ml of water) is then added, and the temperature is fixed at 30° C. The reaction medium is subsequently diluted in 40 ml of water. The final solution is placed in dialysis tubes (cutoff threshold of 8 kD) and is rinsed three times against 51 of 0.9% NaCl and twice against 5 l of distilled water.

The polymer obtained has a random distribution of tryptophan groups on the pendent acid functions. The structure can be represented diagrammatically in the following way

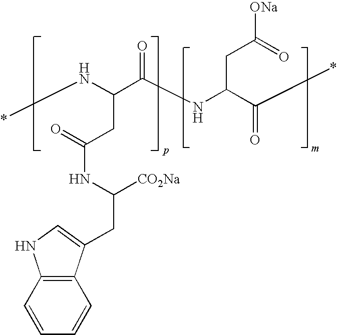

The molar fraction of tryptophan-modified acids is 0.3 according to the ¹H NMR in D₂O (i=0.3). The number p of acid functions bearing a tryptophan is approximately 65. The number m of sodium carboxylate functions is approximately 155. The degree of polymerization n is the sum of m and p.

1. Anionic polyamide functionalized with at least one tryptophan unit, -Trp, said tryptophan unit being linked to the polyamide by an amide function separated from the backbone by a linker arm, wherein it corresponds to the formula below: 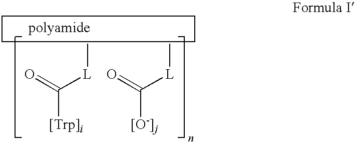
the polyamide being a chain selected from the group constituted of the following polymers 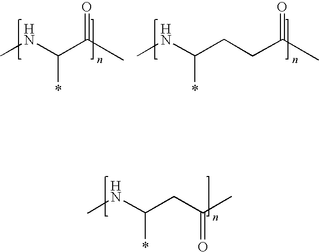
L being selected from the group constituted of a single bond, a —CH₂— group or a —CH₂CH₂— group,
-Trp being an L and/or D tryptophan residue produced from the coupling between the amine of the tryptophan or of a tryptophan derivative, selected from the group constituted of tryptophanol, tryptophanamide and alkali-metal cation salts thereof, and an acid borne by the polyamide,
n is the number of acid functions borne by the polyamide and is between 10 and 10000,
is the molar fraction of acids substituted with -Trp and is between 0.05 and 0.6, and
j is the molar fraction of acids not substituted with -Trp and j is equal to n-i, when the acid is not substituted with -Trp, then the acid is a carboxylate of a cation, preferably a cation of an alkali metal such as Na or K.

2. Anionic polyamide according to claim 1, wherein the polyamide is selected from the poly(alpha-glutamate)s having the formula below:

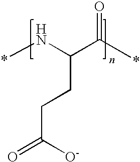

3. Anionic polyamide according to claim 1, wherein the polyamide is selected from the poly(gamma-glutamate)s having the formula below:

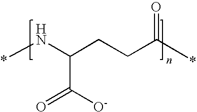

4. Anionic polyamide according to claim 1, wherein the polyamide is selected from the poly(alpha-aspartate)s having the formula below:

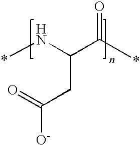

5. Anionic polyamide according to claim 1, wherein the polyamide is selected from the poly(beta-aspartate)s having the formula below:

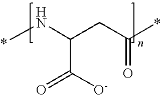

6. Anionic polyamide according to claim 1, wherein the polyamide is a poly(alpha,beta-aspartate).

7. Anionic polyamide according to claim 1, wherein the polyamide has a number of acid functions n of between 10 and 10000.

8. Anionic polyamide according to claim 1, wherein the polyamide has a number of acid functions n of between 10 and 1000.

9. Anionic polyamide according to claim 1, wherein the polyamide has a number of acid functions n of between 10 and 500.

10. Pharmaceutical composition comprising one of the polyamino acids according to claim 1, and at least one active ingredient.

11. Pharmaceutical composition according to claim 1, wherein the active ingredient is selected from the group constituted of proteins, glycoproteins and peptides.
